(19) United States
(12) Patent Application Publication
(10) Pub. No.: US 2012/0064072 A1
(43) Pub. Date: Mar. 15, 2012

(54) Combination Cancer Therapy Comprising Administration of an EGFR Inhibitor and an IGF-1R Inhibitor

(76) Inventors: Maryland Franklin, Lafayette, CO (US); Robert Wild, Carmel, IN (US)

(21) Appl. No.: 13/256,766

(22) PCT Filed: Mar. 18, 2010
(86) PCT No.: PCT/US10/27753

§ 371 Date: Oct. 13, 2011

Related U.S. Application Data
(60) Provisional application No. 61/161,216, filed on Mar. 18, 2009.

(51) Int. Cl. A61K 31/497; A61K 31/5377; A61P 35/00; A61K 39/395

(52) U.S. Cl. 424/133.1; 514/255.05; 424/143.1; 514/234.5; 424/142.1; 514/233.2

(57) ABSTRACT

A method of treating cancer comprising: (a) identifying a patient having cancer that initially responded to IRS1 agent therapy and that has resumed progression; (b) administering an effective regimen comprising the same or a different IRS1 agent and an IGF-1 R inhibitor administered together or sequentially.

### BACKGROUND

Over-expression of the epidermal growth factor receptor (EGFR) kinase, or its ligand TGF-alpha, is frequently associated with many cancers, including breast, lung, colorectal, head and neck cancers (Salomon D. S., et al. (1995) Crit. Rev. Oncol. Hematol. 19:183-232; Wells, A. (2000) Signal, 1:4-11), glioblastomas, and astrocytomas, and is believed to contribute to the malignant growth of these tumors. A specific deletion-mutation in the EGFR gene has also been found to increase cellular tumorigenicity (Halatsch, M-E. et al. (2000) J. Neurosurg. 92:297-305; Archer, G. E. et al. (1999) Clin. Cancer Res. 5:2646-2652). Activation of EGFR stimulated signaling pathways promote multiple processes that are potentially cancer-promoting, e.g., proliferation, angiogenesis, cell motility and invasion, decreased apoptosis and induction of drug resistance.

The development for use as anti-tumor agents of compounds that directly inhibit the kinase activity of the EGFR, as well as antibodies that reduce EGFR kinase activity by blocking EGFR activation, are areas of intense research effort (de Bono J. S., and Rowinsky, E. K. (2002) Trends in Mol. Medicine 8:S19-S26; Dancey, J. and Sausville, E. A. (2003) Nature Rev. Drug Discovery 2:92-313).

Several studies have demonstrated or disclosed that some EGFR kinase inhibitors can improve tumor cell or neoplasia killing when used in combination with certain other anti-cancer or chemotherapeutic agents or treatments (e.g., Raben, D. et al. (2002) Semin. Oncol. 29:37-46; Herbst, R. S. et al. (2001) Expert Opin. Biol. Ther. 1:719-732; Magne, N et al. (2003) Clin. Can. Res. 9:4735-4732; Magne, N. et al. (2002) British Journal of Cancer 86:819-827; Torrance, C. J. et al. (2000) Nature Med. 6:1024-1028; Gupta, R. A. and DuBois, R. N. (2000) Nature Med. 6:974-975; Tortora, et al. (2003) Clin. Cancer Res. 9:1566-1572; Solomon, B. et al (2003) Int. J. Radiat. Oncol. Biol. Phys. 55:713-723; Krishnan, S. et al. (2003) Frontiers in Bioscience 8, e1-13; Huang, S et al. (1999) Cancer Res. 59:1935-1940; Contessa, J. N. et al. (1999) Clin. Cancer Res. 5:405-411; Li, M. et al. Clin. (2002) Cancer Res. 8:3570-3578; Ciardiello, F. et al. (2003) Clin. Cancer Res. 9:1546-1556; Ciardiello, F. et al. (2000) Clin. Cancer Res. 6:3739-3747; Grunwald, V. and Hidalgo, M. (2003) J. Nat. Cancer Inst. 95:851-867; Seymour L. (2003) Current Opin. Investig. Drugs 4(6):658-666; Khalil, M. Y. et al. (2003) Expert Rev. Anticancer Ther. 3:367-380; Bulgaru, A. M. et al. (2003) Expert Rev. Anticancer Ther. 3:269-279; Dancey, J. and Sausville, E. A. (2003) Nature Rev. Drug Discovery 2:92-313; Kim, E. S. et al. (2001) Current Opinion Oncol. 13:506-513; Arteaga, C. L. and Johnson, D. H. (2001) Current Opinion Oncol. 13:491-498; Ciardiello, F. et al. (2000) Clin. Cancer Res. 6:2053-2063; US2003/0108545; US2002/0076408; US2003/0157104; WO99/60023; WO01/12227; WO02/055106; WO03/088971; WO01/34574; WO01/76586; WO02/05791; and WO02/089842).

Erlotinib (e.g. erlotinib HCl, also known as TARCEVA® or OSI-774) is an orally available inhibitor of EGFR kinase. In vitro, erlotinib has demonstrated substantial inhibitory activity against EGFR kinase in a number of human tumor cell lines, including colorectal and breast cancer (Moyer J. D. et al. (1997) Cancer Res. 57:4838), and preclinical evaluation has demonstrated activity against a number of EGFR-expressing human tumor xenografts (Pollack, V. A. et al (1999) J. Pharmacol. Exp. Ther. 291:739). More recently, erlotinib has demonstrated promising activity in phase I and II trials in a number of indications, including head and neck cancer (Soulieres, D., et al. (2004) J. Clin. Oncol. 22:77), NSCLC (Perez-Soler R, et al. (2001) Proc. Am. Soc. Clin. Oncol. 20:310a, abstract 1235), CRC (Oza, M., et al. (2003) Proc. Am. Soc. Clin. Oncol. 22:196a, abstract 785) and MBC (Winer, E., et al. (2002) Breast Cancer Res. Treat. 76:5115a, abstract 445). In a phase III trial, erlotinib monotherapy significantly prolonged survival, delayed disease progression and delayed worsening of lung cancer-related symptoms in patients with advanced, treatment-refractory NSCLC (Shepherd, F. et al. (2005) N. Engl. J. Med. 353(2):123-132). While most of the clinical trial data for erlotinib relate to its use in NSCLC, preliminary results from phase I/II studies have demonstrated promising activity for erlotinib and capecitabine/erlotinib combination therapy in patients with wide range of human solid tumor types, including CRC (Oza, M., et al. (2003) Proc. Am. Soc. Clin. Oncol. 22:196a, abstract 785) and MBC (Jones, R. J., et al. (2003) Proc. Am. Soc. Clin. Oncol. 22:45a, abstract 180). In November 2004 the U.S. Food and Drug Administration (FDA) approved TARCEVA® for the treatment of patients with locally advanced or metastatic non-small cell lung cancer (NSCLC) after failure of at least one prior chemotherapy regimen. TARCEVA® is the only drug in the epidermal growth factor receptor (EGFR) class to demonstrate in a Phase III clinical trial an increase in survival in advanced NSCLC patients.

Target-specific therapeutic approaches, such as erlotinib, are generally associated with reduced toxicity compared with conventional cytotoxic agents, and therefore lend themselves to use in combination regimens. Promising results have been observed in phase I/II studies of erlotinib in combination with bevacizumab (Mininberg, E. D., et al. (2003) Proc. Am. Soc. Clin. Oncol. 22:627a, abstract 2521) and gemcitabine (Dragovich, T., (2003) Proc. Am. Soc. Clin. Oncol. 22:223a, abstract 895). Recent data in NSCLC phase III trials have shown that first-line erlotinib or gefitinib in combination with standard chemotherapy did not improve survival (Gatzemeier, U., (2004) Proc. Am. Soc. Clin. Oncol. 23:617 (Abstract 7010); Herbst, R. S., (2004) Proc. Am. Soc. Clin. Oncol. 23:617 (Abstract 7011); Giaccone, G., et al. (2004) J. Clin. Oncol. 22:777; Herbst, R., et al. (2004) J. Clin. Oncol. 22:785). However, pancreatic cancer phase III trials have shown that first-line erlotinib in combination with gemcitabine did improve survival (OSI Pharmaceuticals/Genentech/Roche Pharmaceuticals Press Release, Sep. 20, 2004). Therefore, the combination of EGFR inhibitors with other anti-cancer agents permits enhanced therapeutic treatment to tumor cells.

Growth factors acting through receptor tyrosine kinases (RTKs) drive tumor initiation and progression by accelerating cell proliferation and promoting cell survival. The RTKs for epidermal growth factor (EGF) and insulin-like growth factor (IGF) contribute to tumorigenesis for a multitude of tumor types including non-small cell lung cancer (NSCLC), colorectal, pancreatic, and breast tumors (Holbro, T., and Hynes, N. E. (2004). ErbB receptors: directing key signaling networks throughout life. Annu Rev Pharmacol Toxicol 44, 195-217; Kurmasheva, R. T., and Houghton, P. J. (2006). IGF-I mediated survival pathways in normal and malignant cells. Biochim Biophys Acta 1766, 1-22; Levitzki, A. (2003). EGF receptor as a therapeutic target. Lung Cancer 41 Suppl 1, S9-14; Roskoski, R., Jr. (2004). The ErbB/HER receptor protein-tyrosine kinases and cancer. Biochem Biophys Res Commun 319, 1-11.) Tumor cells can exhibit redundancy surrounding RTKs that contributes to de novo resistance to a single RTK inhibitor, and crosstalk between RTKs can confer acquired resistance whereby the inhibition of one RTK is compensated by enhanced activity through an alternative RTK.

It has been shown that IGF-1R signaling is associated with acquired resistance of cancer cells to chemo or radiation therapies, and molecular targeted therapies including epidermal growth factor receptor (EGFR) inhibition. Indeed, it has recently been shown that in several different cancer types the efficacy of EGFR and ErbB2 signal transduction inhibitors could be acutely attenuated by IGF-1R activation of the PI3-kinase/Akt pathway (Chakravarti, A., Loeffler, J. S., and Dyson, N. J. (2002). Insulin-like growth factor receptor I mediates resistance to anti-epidermal growth factor receptor therapy in primary human glioblastoma cells through continued activation of phosphoinositide 3-kinase signaling. Cancer research 62, 200-207; Jones, H. E., Goddard, L., Gee, J. M., Hiscox, S., Rubini, M., Barrow, D., Knowlden, J. M., Williams, S., Wakeling, A. E., and Nicholson, R. I. (2004). Insulin-like growth factor-I receptor signaling and acquired resistance to gefitinib (ZD1839; Iressa) in human breast and prostate cancer cells. Endocr Relat Cancer 11, 793-814; Lu, Y., Zi, X., Zhao, Y., Mascarenhas, D., and Pollak, M. (2001). Insulin-like growth factor-I receptor signaling and resistance to trastuzumab (Herceptin). Journal of the National Cancer Institute 93, 1852-1857; Nahta, R., Yuan, L. X., Zhang, B., Kobayashi, R., and Esteva, F. J. (2005). Insulin-like growth factor-I receptor/human epidermal growth factor receptor 2 heterodimerization contributes to trastuzumab resistance of breast cancer cells. Cancer research 65, 11118-11128). For instance, IGF-1R activation correlates with acquired resistance of breast and prostate cancer cells to EGFR inhibition (Jones et al., 2004). IGF-1R has also been shown to mediate resistance to anti-EGFR therapies in glioblastoma, colorectal, and NSCLC tumor cells (Chakravarti et al., 2002; Liu et al., 2001; Jones et al., 2004; Morgillo et al., 2006; Hurbin et al., 2003; Knowlden et al., 2005).

US2006/0235031 refers to 6,6-bicyclic ring substituted heterobicyclic protein kinase inhibitors as IFG1R inhibitors and uses thereof, including for treating cancer. US2003/0114467; US2003/0153752; and US2005/0037999 refer to pyrazolo- and pyrrolo-pyrimidines and uses thereof, including for cancer treatment, and generally refer to various combinations with other anticancer agents. US2005/0153966 refers to heterocyclic compounds said to be kinase inhibitors and uses thereof, including for cancer treatment. US2004/0180911 refers to pyrimidine derivatives and uses thereof, including for tumors and proliferative diseases, and states that the compounds can be used in combination with other chemotherapy drugs. WO2004/056830 refers to pyrrolopyrimidine derivatives and uses thereof, including for cancer treatment, and states that the compounds can be used in combination with other anticancer agents.

Valeriote et al., Cancer Chemotherapy Reports, 59(5), 895-900 (1975), states that “extensive literature describing additivity and synergism in anticancer agents exists.” US2004/0106605 is entitled “Synergistic Methods and Compositions for Treating Cancer,” and generally refers to combinations of IGF1R inhibitors with EGFR inhibitors. US2008/0267957 and US2008/0014200 disclose methods of treatment that include administering both an IGF-1R inhibitor and an EGFR inhibitor. These publications are incorporated herein in their entireties for all purposes, including the particular IGF-1R inhibitors, EGFR inhibitors, underlying mechanistic information, and methods of treatment.

Also noted are Harris et al., Diseases of the Breast, p. 1193 (2005); Ueda et al., Modern Path., 19, 788-796 (2006); Wilsbacher et al., J. Biol. Chem., 283, 35, 23721-30 (2008); Science Daily Jun. 25, 2008 (http://www.sciencedaily.com/releases/2008/06/080624135934.htm; accessed Jan. 13, 2009); Takahari et al., Oncology, 76, 42-48 (2009); Erlotinib With or Without IMC-A12 (clinicaltrials.gov/ct2/show/NCT00778167?show_desc=Y; accessed Jan. 13, 2009); Riely et al., Clin. Cancer Res., 13(17) (September 2007); In vitro studies have been presented to support the hypothesis that an EGFR and IGF-1R inhibitor combination could synergistically inhibit proliferation and potentially drive apoptosis in early stage tumors with an epithelial phenotype—Barr et al., Clin. Exp. Metastasis, 25:685-693 (2008); M. Höpfner, Free University Berlin Dissertations Online (2007) (www.diss.fuberlin.de/diss/receive/fudiss_thesis_{—}000000002588?lang=en; accessed Jan. 13, 2009).

Unfortunately, not all subjects respond to Tarceva/erlotinib or other EGFR inhibitors. Moreover, most responders eventually progress after a period of treatment. Standard protocol may call for cessation of EGFR inhibitor treatment when progression occurs. Thus, there is a need for improved cancer treatments, including treatments through which the effectiveness duration of an EGFR inhibitor is prolonged or extended, such as by increasing the time of progression-free survival on the EGFR inhibitor, such as by the addition of another small molecule therapeutic agent to the treatment regimen.

### SUMMARY

The present invention includes methods and compositions for treating cancers, human cancer, tumors, and tumor metastases. In particular, the present invention includes treatment of subjects including human cancer patients with an effective regimen comprising both of at least one IGF-1R protein kinase inhibitor and at least one agent that inhibits serine phosphorylation of IRS1 (IRS1 agent), such as an EGFR inhibitor. In some preferred embodiments, the patient or subject selected for treatment is one whose cancer initially responds to the IRS1 agent, followed by eventual cancer progression (refractory).

In some embodiments, the IGF-1R inhibitor comprises a small-molecule tyrosine kinase inhibitor (TKI). In some embodiments, the IGF-1R inhibitor comprises a compound or salt thereof as described in US2006/0235031. In some embodiments, the IGF-1R inhibitor comprises OSI-906, i.e., cis-3-[8-amino-1-(2-phenyl-quinolin-7-yl)-imidazo[1,5-a]pyrazin-3-yl]-1-methyl-cyclobutanol or a pharmaceutically acceptable salt thereof.

In some embodiments, the initially effective IRS1 agent is erlotinib or gefitinib. In some embodiments, the IRS1 agent used with the IGF-1R inhibitor is erlotinib or gefitinib.

In some embodiments, the cancer is initially treatable, either partially or completely, with EGFR kinase inhibitor therapy. In some embodiments, the cancer is NSCLC. In some embodiments, the cancer is selected from lung, pancreatic, head and neck, breast, adrenocortical carcinoma (ACC), colorectal, ovarian, renal cell, bladder, glioblastoma, astrocytoma, or neuroblastoma.

In some embodiments, the administration of the IGF-1R inhibitor and the EGFR inhibitor is additive or is synergistic.

In some embodiments, continuation of IRS1 agent treatment with the addition of the IGF-1R inhibitor at or around the time of progression provides improved progression-free survival time or other measurable benefit.

In some embodiments, an EGFR inhibitor and the IGF-1R inhibitor are administered sequentially. In some embodiments, an EGFR inhibitor and the IGF-1R inhibitor are administered together. In some embodiments, additional active agent(s) are administered that improve survival time and/or overall success of the regimen.

In some embodiments, the invention includes a pharmaceutical composition and the manufacture of a medicament(s) for use in practicing the methods herein.

### BRIEF DESCRIPTION OF DRAWINGS

FIG. 1: GEO Model RTK array analysis for untreated mice and mice following 18 days erlotinib treatment (100 mg/kg/day) showing median pixel density.

FIG. 2: GEO Model Western blot analysis of AKT and ERK.

FIG. 3: GEO model TBP study design.

FIG. 4: GEO model TBP data (days to tumor doubling).

FIG. 5: H292 Model RTK array analysis for untreated mice and mice following 18 days erlotinib treatment (100 mg/kg/day) showing mean pixel density as a percent of phosphotyrosine loading control samples.

FIG. 6: H292 Model Western blot analysis of AKT and ERK.

FIG. 7: H292 model TBP study design.

FIG. 8: H292 model initial erlotinib treatment (100 mg/kg/day).

FIG. 9: First trial: H292 model TBP data (tumor volume).

FIG. 10: First trial: H292 model TBP data statistical Kaplan-Meier analysis of data for time to one doubling.

FIG. 11: First trial: H292 model treatment beyond progression data statistical Kaplan-Meier analysis of data for time to two doublings.

FIG. 12: H292 model frontline combination data (tumor volume).

FIG. 13: Second trial: H292 model TBP data (tumor volume).

FIG. 14: Second trial: H292 model TBP data statistical Kaplan-Meier analysis of data for time to one doubling.

FIG. 15: Second trial: H292 model treatment beyond progression data statistical Kaplan-Meier analysis of data for time to two doublings.

### DETAILED DESCRIPTION

Many patients with NSCLC who initially respond to EGFR kinase inhibitor therapy later develop progressive disease, and become refractory to EGFR kinase inhibitor therapy. It will be appreciated by one of skill in the medical arts that there are many reasons why a patient may become refractory to treatment with an EGFR kinase inhibitor as a single agent, one of which is that the tumor cells of the patient develop insensitivity to inhibition by the tested EGFR kinase inhibitor. It is also possible that a patient may become refractory to treatment with one type of EGFR kinase inhibitor, but be sensitive to treatment with another type of EGFR kinase inhibitor.

Continuation of treatment in patients with initial response to EGFR kinase inhibitor therapy followed by progressive disease may be beneficial even when a new treatment is initiated. The present invention provides for the continuation of therapy with the addition of an IGF-1R kinase inhibitor. Thus, continuation of EGFR (or other IRS1 agent) treatment following progressive disease can be beneficial even when the new treatment is initiated.

The present invention further provides a method for reducing the side effects caused by the treatment of tumors or tumor metastases in a refractory patient with an EGFR kinase inhibitor or an IGF-1R kinase inhibitor, comprising administering to the patient simultaneously or sequentially a therapeutically effective amount of a combination of an EGFR kinase inhibitor and an IGF-1R kinase inhibitor, in amounts that are effective to produce an additive, or a synergistic antitumor effect, and that are effective at inhibiting tumor growth.

The present invention further provides a method for the treatment of refractory cancer, comprising administering to a subject in need of such treatment an effective regimen comprising (i) an effective or sub-therapeutic first amount of an EGFR kinase inhibitor, or a pharmaceutically acceptable salt thereof; and (ii) an effective or sub-therapeutic second amount of an IGF-1R kinase inhibitor.

In some embodiments, the invention provides anti-cancer combination therapies that reduce the dosages for individual therapeutic components required for efficacy, thereby decreasing side effects, while maintaining or increasing therapeutic value, such as in terms of survival time.

### Subjects and Indications

In some embodiments, the subject or patient is one whose cancer has responded to an IRS1 agent such as an EGFR kinase inhibitor, and subsequently progresses. Thus the initial IRS1 agent is any initially effective agent for the patient's condition. The IRS1 agent used going forward in the regimen of this invention can be the same or a different inhibitor.

In some embodiments, the patient is a human in need of treatment for cancer or other forms of abnormal cell growth. The cancer is preferably any cancer that is treatable, either partially or completely, by administration of an IRS1 agent such as an EGFR kinase inhibitor.

In some embodiments, the cancer is selected from colorectal cancer, non-small cell lung carcinoma (NSCLC), adrenocortical carcinoma (ACC), pancreatic cancer, head and neck cancer, breast cancer, or neuroblastoma. The cancer may also be, for example: NSCL cancer, breast cancer, colon cancer, pancreatic cancer, lung cancer, bronchioloalveolar cell lung cancer, bone cancer, skin cancer, cancer of the head or neck, cutaneous or intraocular melanoma, uterine cancer, ovarian cancer, rectal cancer, cancer of the anal region, stomach cancer, gastric cancer, uterine cancer, carcinoma of the fallopian tubes, carcinoma of the endometrium, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the ureter, cancer of the kidney, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions.

### IRS1 Agent

According to the invention, agents that inhibit serine phosphorylation of IRS1 include inhibitors of the MAPK pathway, including for example EGFR inhibitors, MEK inhibitors, Ras inhibitors, Raf inhibitors, and PKC inhibitors.

The IRS agent is preferably an EGFR kinase inhibitor. As used herein, the term “EGFR kinase inhibitor” refers to any effective EGFR kinase inhibitor that is currently known in the art or that will be identified in the future, and includes any chemical entity that, upon administration to a patient, results in inhibition of a biological activity associated with activation of the EGF receptor in the patient, including any of the downstream biological effects otherwise resulting from the binding to EGFR of its natural ligand. Such EGFR kinase inhibitors include any agent that can block EGFR activation or any of the downstream biological effects of EGFR activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the EGF receptor, thereby making the receptor inaccessible to its natural ligand so that its normal biological activity is prevented or reduced. Alternatively, such an inhibitor can act by modulating the dimerization of EGFR polypeptides, or interaction of EGFR polypeptide with other proteins, or enhance ubiquitination and endocytotic degradation of EGFR. EGFR kinase inhibitors include but are not limited to low molecular weight or small molecule inhibitors, antibodies or antibody fragments, peptide or RNA aptamers, antisense constructs, small inhibitory RNAs (i.e. RNA interference by dsRNA; RNAi), and ribozymes. In certain embodiments, the EGFR kinase inhibitor is a small organic molecule or an antibody that binds specifically to the human EGFR.

EGFR kinase inhibitors include, for example quinazoline EGFR kinase inhibitors, pyrido-pyrimidine EGFR kinase inhibitors, pyrimido-pyrimidine EGFR kinase inhibitors, pyrrolo-pyrimidine EGFR kinase inhibitors, pyrazolo-pyrimidine EGFR kinase inhibitors, phenylamino-pyrimidine EGFR kinase inhibitors, oxindole EGFR kinase inhibitors, indolocarbazole EGFR kinase inhibitors, phthalazine EGFR kinase inhibitors, isoflavone EGFR kinase inhibitors, quinalone EGFR kinase inhibitors, and tyrphostin EGFR kinase inhibitors, such as those described in the following patent publications, and all pharmaceutically acceptable salts and solvates of said EGFR kinase inhibitors: International Patent Publication Nos. WO96/33980, WO96/30347, WO97/30034, WO97/30044, WO97/38994, WO97/49688, WO98/02434, WO97/38983, WO95/19774, WO95/19970, WO97/13771, WO98/02437, WO98/02438, WO97/32881, WO98/33798, WO97/32880, WO97/03288, WO97/02266, WO97/27199, WO98/07726, WO97/34895, WO96/31510, WO98/14449, WO98/14450, WO98/14451, WO95/09847, WO97/19065, WO98/17662, WO99/35146, WO99/35132, WO99/07701, WO92/20642; EP520722, EP566226, EP787772, EP837063, EP682027; U.S. Pat. No. 5,747,498, U.S. Pat. No. 5,789,427, U.S. Pat. No. 5,650,415, U.S. Pat. No. 5,656,643, U.S. Pat. No. 6,900,221, and DE19629652. Additional non-limiting examples of low molecular weight EGFR kinase inhibitors include any of the EGFR kinase inhibitors described in Traxler, P., 1998, Exp. Opin. Ther. Patents 8(12):1599-1625.

Specific examples of low molecular weight EGFR kinase inhibitors that can be used according to the present invention include [6,7-bis(2-methoxyethoxy)-4-quinazolin-4-yl]-(3-ethynylphenyl)amine (also known as OSI-774, erlotinib, or TARCEVA® (erlotinib HCl); OSI Pharmaceuticals/Genentech/Roche) (U.S. Pat. No. 5,747,498; WO01/34574, and Moyer, J. D. et al. (1997) Cancer Res. 57:4838-4848); CI-1033 (formerly known as PD183805; Pfizer) (Sherwood et al., 1999, Proc. Am. Assoc. Cancer Res. 40:723); PD-158780 (Pfizer); AG-1478 (University of California); CGP-59326 (Novartis); PKI-166 (Novartis); EKB-569 (Wyeth); GW-2016 (also known as GW-572016 or lapatinib ditosylate; GSK); and gefitinib (also known as ZD1839 or IRESSA™; Astrazeneca) (Woodburn et al., 1997, Proc. Am. Assoc. Cancer Res. 38:633). EGFR kinase inhibitors also include, for example multi-kinase inhibitors that have activity on EGFR kinase, i.e., inhibitors that inhibit EGFR kinase and one or more additional kinases. Examples of such compounds include the EGFR and HER2 inhibitor CI-1033 (formerly known as PD183805; Pfizer); the EGFR and HER2 inhibitor GW-2016 (also known as GW-572016 or lapatinib ditosylate; GSK); the EGFR and JAK 2/3 inhibitor AG490 (a tyrphostin); the EGFR and HER2 inhibitor ARRY-334543 (Array BioPharma); BIBW-2992, an irreversible dual EGFR/HER2 kinase inhibitor (Boehringer Ingelheim Corp.); the EGFR and HER2 inhibitor EKB-569 (Wyeth); the VEGF-R2 and EGFR inhibitor ZD6474 (also known as ZACTIMA™; AstraZeneca Pharmaceuticals), and the EGFR and HER2 inhibitor BMS-599626 (Bristol-Myers Squibb).

Antibody-based EGFR kinase inhibitors include any anti-EGFR antibody or antibody fragment that can partially or completely block EGFR activation by its natural ligand. Non-limiting examples of antibody-based EGFR kinase inhibitors include those described in Modjtahedi, H., et al., 1993, Br. J. Cancer 67:247-253; Teramoto, T., et al., 1996, Cancer 77:639-645; Goldstein et al., 1995, Clin. Cancer Res. 1:1311-1318; Huang, S. M., et al., 1999, Cancer Res. 15:59(8):1935-40; and Yang, X., et al., 1999, Cancer Res. 59:1236-1243. Thus, the EGFR kinase inhibitor can be the monoclonal antibody Mab E7.6.3 (Yang, X. D. et al. (1999) Cancer Res. 59:1236-43), or Mab C225 (ATCC Accession No. HB-8508), or an antibody or antibody fragment having the binding specificity thereof. Suitable monoclonal antibody EGFR kinase inhibitors include, but are not limited to, IMC-C225 (also known as cetuximab or ERBITUX™; Imclone Systems), ABX-EGF (Abgenix), EMD 72000 (Merck KgaA, Darmstadt), RH3 (York Medical Bioscience Inc.), and MDX-447 (Medarex/Merck KgaA). Additional antibody-based EGFR kinase inhibitors or KIT kinase inhibitors can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against EGFR or KIT can be prepared and isolated using any technique that provides for the production of antibody molecules by continuous cell lines in culture. Techniques for production and isolation include but are not limited to the hybridoma technique originally described by Kohler and Milstein (Nature, 1975, 256: 495-497); the human B-cell hybridoma technique (Kosbor et al., 1983, Immunology Today 4:72; Cote et al., 1983, Proc. Natl. Acad. Sci. USA 80: 2026-2030); and the EBV-hybridoma technique (Cole et al., 1985, Monoclonal Antibodies and Cancer Therapy, Alan R. Liss, Inc., pp. 77-96).

Alternatively, techniques described for the production of single chain antibodies (see, e.g., U.S. Pat. No. 4,946,778) can be adapted to produce anti-EGFR or anti-Kit single chain antibodies. Antibody-based EGFR kinase inhibitors or KIT kinase inhibitors useful in practicing the present invention also include anti-EGFR or anti-Kit antibody fragments including but not limited to F(ab′)₂ fragments, which can be generated by pepsin digestion of an intact antibody molecule, and Fab fragments, which can be generated by reducing the disulfide bridges of the F(ab′).sub.2 fragments. Alternatively, Fab and/or scFv expression libraries can be constructed (see, e.g., Huse et al., 1989, Science 246: 1275-1281) to allow rapid identification of fragments having the desired specificity to EGFR or Kit.

Techniques for the production and isolation of monoclonal antibodies and antibody fragments are well-known in the art, and are described in Harlow and Lane, 1988, Antibodies: A Laboratory Manual, Cold Spring Harbor Laboratory, and in J. W. Goding, 1986, Monoclonal Antibodies: Principles and Practice, Academic Press, London. Humanized anti-EGFR antibodies and antibody fragments can also be prepared according to known techniques such as those described in Vaughn, T. J. et al., 1998, Nature Biotech. 16:535-539 and references cited therein, and such antibodies or fragments thereof are also useful in practicing the present invention.

### IGF-1R Inhibitor

The IGF-1R kinase inhibitor is any such agent that enhances the effect of continuing the IRS1 agent. In some embodiments, the IGF-1R kinase inhibitor is a small molecule organic compound or salt thereof. For example, the IGF-1R kinase inhibitor can be any compound or salt thereof as described in US2006/0235031 or in US2006/0084654, both of which are incorporated herein by reference in their entireties for all purposes. The IGF-1R kinase inhibitor can be OSI-906 ((cis-3-[8-amino-1-(2-phenyl-quinolin-7-yl)-imidazo[1,5-a]pyrazin-3-yl]-1-methyl-cyclobutanol).

### Compositions

The active agents can be administered with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, etc. Oral pharmaceutical compositions can be suitably sweetened and/or flavored.

Methods of preparing pharmaceutical compositions comprising an EGFR kinase inhibitor are known in the art, and are described, e.g., in WO01/34574. In view of the teaching of the present invention, methods of preparing pharmaceutical compositions comprising an EGFR kinase inhibitor and/or an IGF-1R kinase inhibitor will be apparent from the above-cited publications and from other known references, such as Remington's Pharmaceutical Sciences, Mack Publishing Company, Easton, Pa., 18^{th} edition (1990).

For oral administration of EGFR kinase inhibitors or IGF-1R kinase inhibitor, tablets containing one or both of the active agents are combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the EGFR or IGF-1R kinase inhibitor may be combined with various sweetening or flavoring agents, coloring matter or dyes, and, if so desired, emulsifying and/or suspending agents as well, together with such diluents as water, ethanol, propylene glycol, glycerin and various like combinations thereof.

### Dosing and Administration

The exact manner of administering a therapeutically effective regimen can depend upon a particular patient and the condition. The administration, including dosage, combination with other anti-cancer agents, timing and frequency of administration, and the like, may be affected by the diagnosis of a subject or patient's likely responsiveness as well as the patient's condition and history.

In some embodiments of the methods of this invention, an IGF-1R kinase inhibitor is administered at the same time as an EGFR kinase inhibitor. In some embodiments of the methods of this invention, an IGF-1R kinase inhibitor is administered prior to the EGFR kinase inhibitor. In other embodiments of the methods of this invention, an IGF-1R kinase inhibitor is administered after the EGFR kinase inhibitor. In another embodiment of the methods of this invention, an IGF-1R kinase inhibitor is pre-administered prior to administration of a combination of an EGFR kinase inhibitor and an IGF-1R kinase inhibitor.

Dosage levels for the compounds of the combination of this invention can be approximately as described herein, or as described in the art for these compounds. It is understood, however, that the exact dose level for any particular patient can depend upon a variety of factors including the age, body weight, general health, sex, diet, time of administration, route of administration, rate of excretion, drug combination and the severity of the particular disease undergoing therapy.

Dosing and administration may be dictated at least in part by the PK and PD properties of the agents alone and/or in combination.

The invention provides anti-cancer combination therapies that reduce the dosages for individual components required for efficacy, thereby decreasing side effects associated with each agent, while maintaining or increasing therapeutic value. This can be optimized based upon the active agents being used.

The amount of EGFR kinase inhibitor administered and the timing of EGFR kinase inhibitor administration will depend on the type (species, gender, age, weight, etc.) and condition of the patient being treated, the severity of the disease or condition being treated, and on the route of administration. For example, in addition to the descriptions above, small molecule EGFR kinase inhibitors can be administered to a patient in doses ranging from 0.001 to 100 mg/kg of body weight per day or per week in single or divided doses, or by continuous infusion (see for example WO 01/34574). In particular, erlotinib HCl can be administered to a patient in doses ranging from 5-200 mg per day, or 100-1600 mg per week, in single or divided doses, or by continuous infusion. A preferred dose is 150 mg/day. Antibody-based EGFR kinase inhibitors, or antisense, RNAi or ribozyme constructs, can be administered to a patient in doses ranging from 0.1 to 100 mg/kg of body weight per day or per week in single or divided doses, or by continuous infusion. In some instances, dosage levels below the lower limit of the aforesaid range may be more than adequate, while in other cases still larger doses may be employed without causing unjustifiably harmful side effects.

Thus, the invention includes method of treating cancer comprising: (a) selecting a patient having cancer that responded to a first IRS1 agent such as EGFR inhibitor therapy and that has resumed progression; (b) administering to the patient an effective regimen comprising (i) at least one second EGFR inhibitor and (ii) at least one IGF-1R inhibitor; wherein the at least one second EGFR inhibitor is the same or different as the agent(s) used in the first EGFR inhibitor therapy, and; wherein (i) and (ii) are administered together or sequentially.

In some embodiments, the IGF-1R inhibitor comprises OSI-906 or a pharmaceutically acceptable salt thereof. In some embodiments thereof, the OSI-906 is administered in an amount of about 0.1 to about 0.7 mg/kg·day, about 0.7 to about 5 mg/kg·day, or about 5 to about 15 mg/kg·day.

In some embodiments, the second EGFR inhibitor comprises a small-molecule non-biologic agent. In some embodiments thereof, the second EGFR inhibitor comprises erlotinib or a pharmaceutically acceptable salt thereof. In some embodiments, the second EGFR inhibitor comprises gefitinib, CI-1033, cetuximab, panitumumab, lapatinib, lapatinib ditosylate, ZACTIMA™, BMS-599626, ARRY-334543, or AG490. In some embodiments, the second EGFR inhibitor comprises a monoclonal antibody.

In some embodiments, the second EGFR inhibitor comprises the same agent(s) used in the first EGFR inhibitor therapy.

In some embodiments of the invention, the method further comprising administering at least one additional active agent.

In some embodiments, the second EGFR and IGF-1R inhibitors behave synergistically. In other embodiments, they behave additively.

In some embodiments, the patient exhibits progression-free survival for at least about two, four, eight, sixteen, or thirty-two weeks from the administration of the first dose of the IGF-1R inhibitor.

In some embodiments, the time to tumor volume doubling is at least about three, six, twelve, or twenty-four weeks from the administration of the first dose of the IGF-1R inhibitor.

### In Vivo Data

This invention will be better understood from the experimental details that follow. However, one skilled in the art will readily appreciate that the specific methods and results discussed are merely illustrative of the invention as described more fully in the claims which follow thereafter, and are not to be considered in any way limited thereto.

The H292 (NSCLC) and GEO (CRC) human tumor xenograft models were used to evaluate the efficacy of EGFR inhibitor (erlotinib) maintenance therapy with and without the addition of IGF-1R inhibitor (OSI-906) therapy following response and subsequent progression on erlotinib monotherapy.

Anti-tumor efficacy was evaluated using mouse xenograft tumor models derived from GEO and H292 cells. Female athymic nude nu/nu CD-1 mice (6-8 wks, 22-29 g) were obtained from Charles River Laboratories (Wilmington, Mass.). Animals were allowed to acclimate for a minimum of one week prior to initiation of a study. Throughout the studies, animals were allowed sterile rodent chow and water ad libitum, and immunocompromised animals were maintained under specific pathogen free conditions. All animal studies were conducted with the approval of the Institutional Animal Care and Use Committee in an American Association for Accreditation of Laboratory Animal Care (AAALAC)-accredited vivarium and in accordance with the Institute of Laboratory Animal Research (Guide for the Care and Use of Laboratory Animals, NIH, Bethesda, Md.). Tumor cells were harvested from cell culture flasks during exponential cell growth, washed twice with sterile PBS, counted and resuspended in PBS to a suitable concentration before s.c. implantation on the right flank of female nu/nu CD-1 mice. Tumors were established to 200+/−50 mm³ in size before randomization into treatment groups of 8 mice each. Body weights were determined twice weekly along with tumor volume {V=[length×(width)²]/2} measurements using Vernier calipers during the study. Tumor growth inhibition (% TGI) was determined by the following formula: % TGI={1−[(Tₜ/T₀)/(Cₜ/C₀)]/1-[C₀/Cₜ]}×100. Tₜ is tumor volume of treated at time t; T₀ is tumor volume of treated at time 0; Cₜ is tumor volume of control at time t; and C₀ is tumor volume of control at time 0. Antitumor activity was defined as a minimum tumor growth inhibition of 50% at the end of treatment. Furthermore, we evaluated the effect of drug treatment on tumor growth delay (GD or T-C value), defined as the difference in time (days) required for the treated tumors (T) to reach 400% of the initial tumor volume compared with those of the control group (C). Cures were excluded from this particular calculation. At the time of tumor progression on single agent erlotinib treatment mice were randomized into additional study groups of 8 mice each. Individual tumor volumes were calculated, as stated above, for all mice throughout the study and the time in days until each mouse doubled its tumor volume from the time of re-randomization was determined by linear regression analysis of the entire data set. Statistical evaluation of the data was determined by Kaplan-Meier Survival analysis.

### RTK Array Analysis—GEO Model

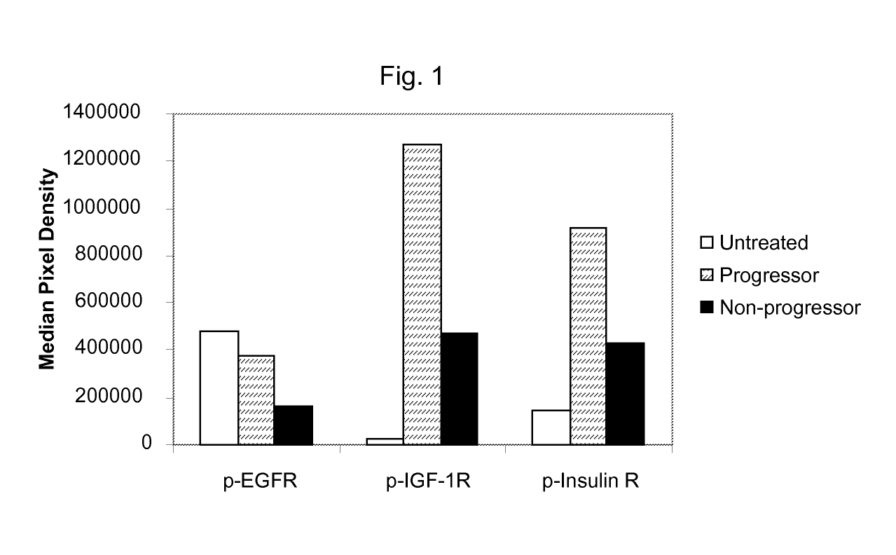

In untreated GEO tumors and tumors treated with erlotinib (100 mg/kg per day) for eighteen days, the levels of pEGFR, pIGF-1R, and pIR were measured in terms of median pixel density, as shown in FIG. 1. All treated tumors exhibited upregulation of pIGF-1R and pIR as compared to untreated tumors. Tumors that initially responded and then progressed showed the greatest upregulation.

### GEO Model Western Blot Analysis of AKT and ERK

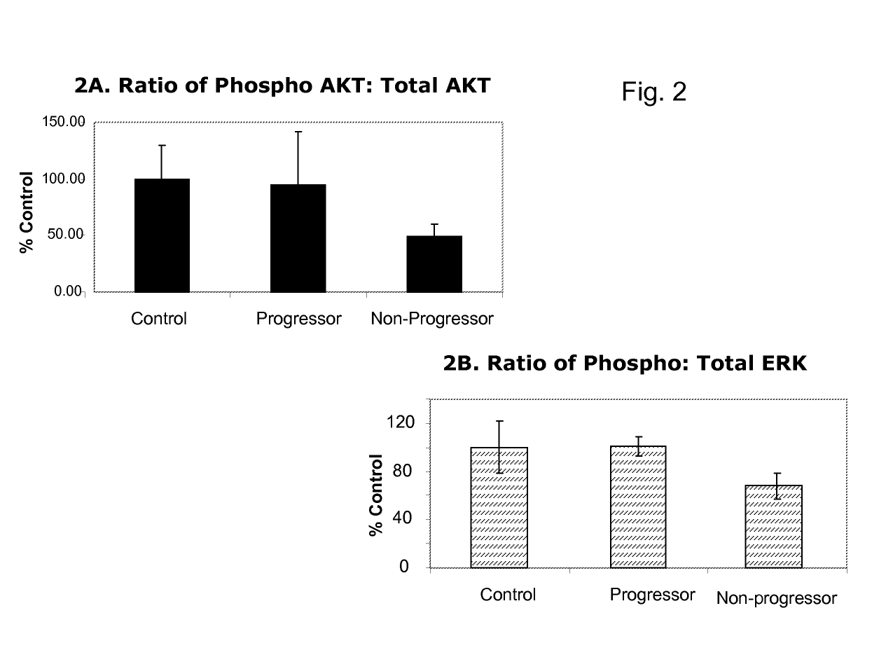

In untreated GEO tumors and tumors treated with erlotinib (100 mg/kg per day) for eighteen days, the levels of total and phosphorylated Akt and Erk were measured and quantitated in terms of the ratio of phosphor to total protein in each sample and then plotted as compared to levels observed in vehicle control treated tumors (assumed to be 100%). Tumors that do not progress on treatment demonstrate continued inhibition of downstream signaling markers (Akt, Erk). Tumors that respond and then progress on treatment are no longer inhibiting these markers, as shown in FIG. 2.

### Treatment Beyond Progression—GEO Model

To run the preclinical treatment beyond progression study GEO cells were harvested from cell culture flasks during exponential cell growth, washed twice with sterile PBS, counted and resuspended in PBS to a suitable concentration before s.c. implantation on the right flank of female nu/nu CD-1 mice. Tumors were established to 200+/−50 mm³ in size before randomization into vehicle control or erlotinib treatment groups.

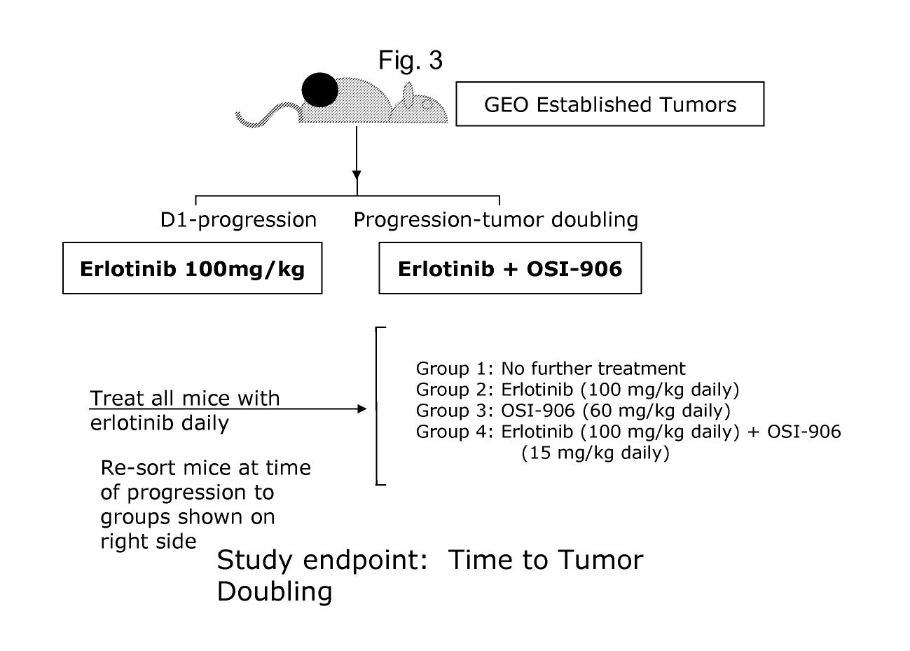

Following 18 days of oral dosing with erlotinib (100 mg/kg per day), mice that initially responded to erlotinib (demonstrated tumor growth inhibition as described above) and then started to have tumor regrowth (progression) while still on erlotinib treatment were randomly re-sorted into one of the following groups, n=8 per group: 1) no further treatment, 2) maintenance of erlotinib treatment (100 mg/kg per day), 3) taken off erlotinib and put on OSI-906 (60 mg/kg per day) treatment, and 4) maintained on erlotinib (100 mg/kg per day) treatment and had OSI-906 (15 mg/kg per day) added to the treatment regimen. This is illustrated schematically in FIG. 3.

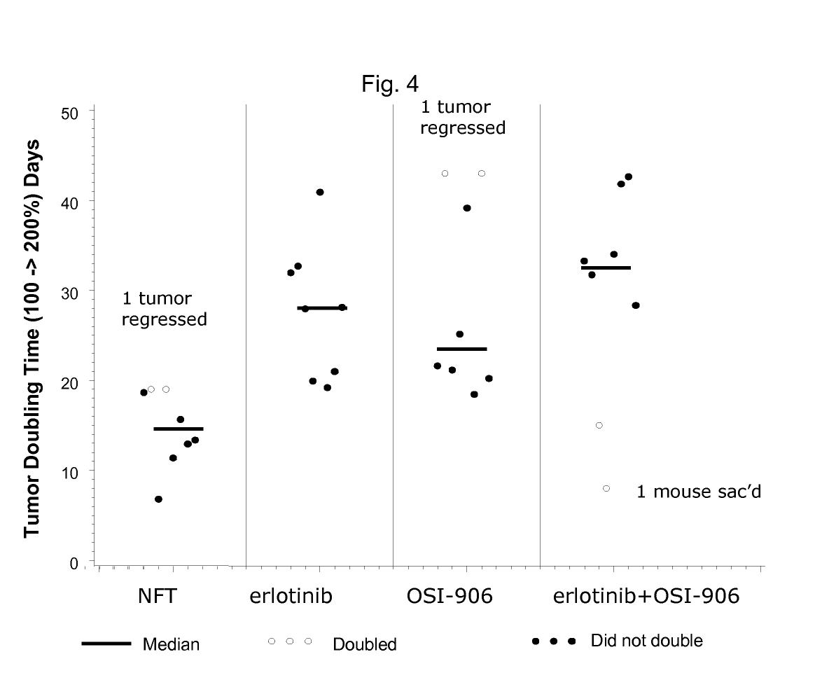

Each animal was maintained on its designated dosing regimen until its tumor volume from the time of re-sort had doubled. See FIG. 4. As shown in FIG. 4, the median time to tumor doubling following the re-sorting was about 28 days for erlotinib maintenance (100 mg/kg per day) and about 32 days for the combination therapy (p=0.0468).

### RTK Array Analysis—H292 Model

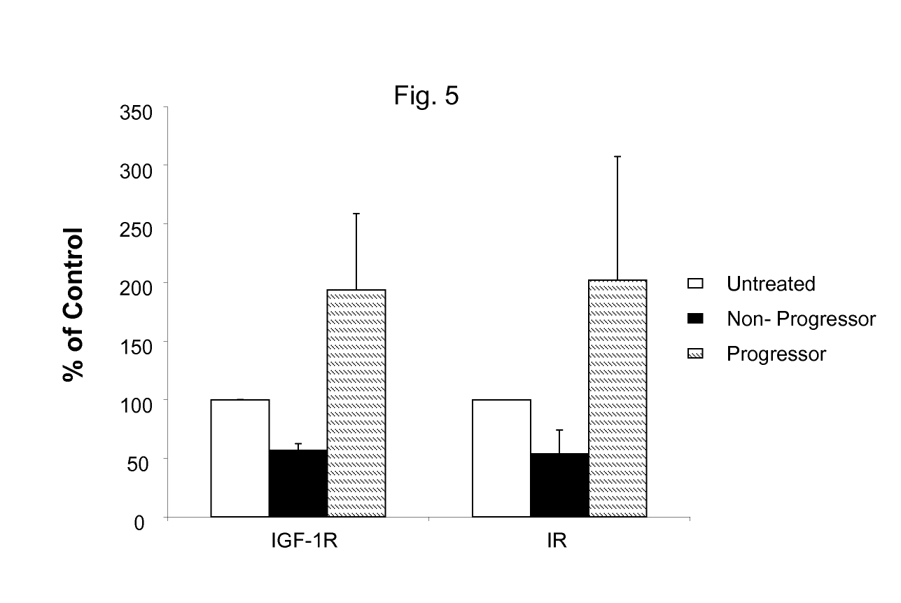

In untreated H292 tumors and tumors treated with erlotinib (100 mg/kg per day) for eighteen days, the levels of pIGF-1R and pIR were measured in terms of pixel density normalized to phosphotyrosine loading control, as shown in FIG. 5. Tumors that initially responded and then progressed (progressor) show upregulation of both pIGF-1R and pIR as compared to untreated tumors or tumors that continue to respond to erlotinib treatment (non-progressor).

### H292 Model Western Blot Analysis of AKT and ERK

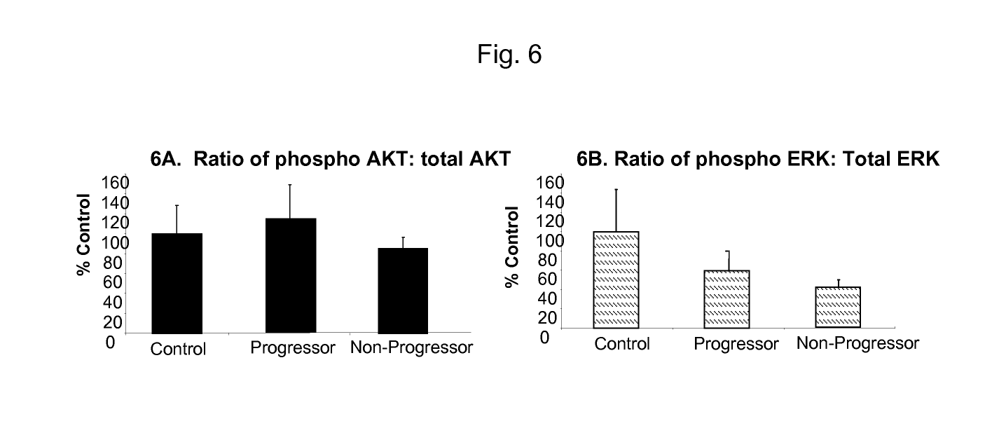

In untreated H292 tumors and tumors treated with erlotinib (100 mg/kg per day) for eighteen days, the levels of total and phosphorylated Akt and Erk were measured and quantitated in terms of the ratio of phosphor to total protein in each sample and then plotted as compared to levels observed in vehicle control treated tumors (assumed to be 100%). Tumors that do not progress on treatment demonstrate continued inhibition of downstream signaling markers (Akt, Erk). Tumors that respond and then progress on treatment are no longer inhibiting these markers, as shown in FIG. 6.

### Treatment Beyond Progression (TBP)—H292 Model

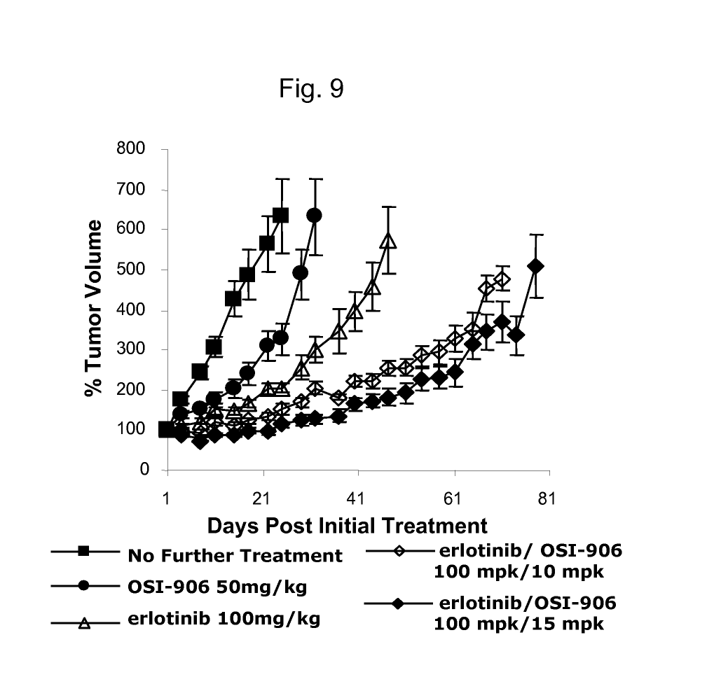

In two separate experiments (FIGS. 9-11 and FIGS. 13-15), NCI-H292 cells were harvested from cell culture flasks during exponential cell growth, washed twice with sterile PBS, counted and resuspended in PBS to a suitable concentration before s.c. implantation on the right flank of female nu/nu CD-1 mice. Tumors were established to 200+/−50 mm³ in size before randomization into vehicle control or erlotinib treatment groups.

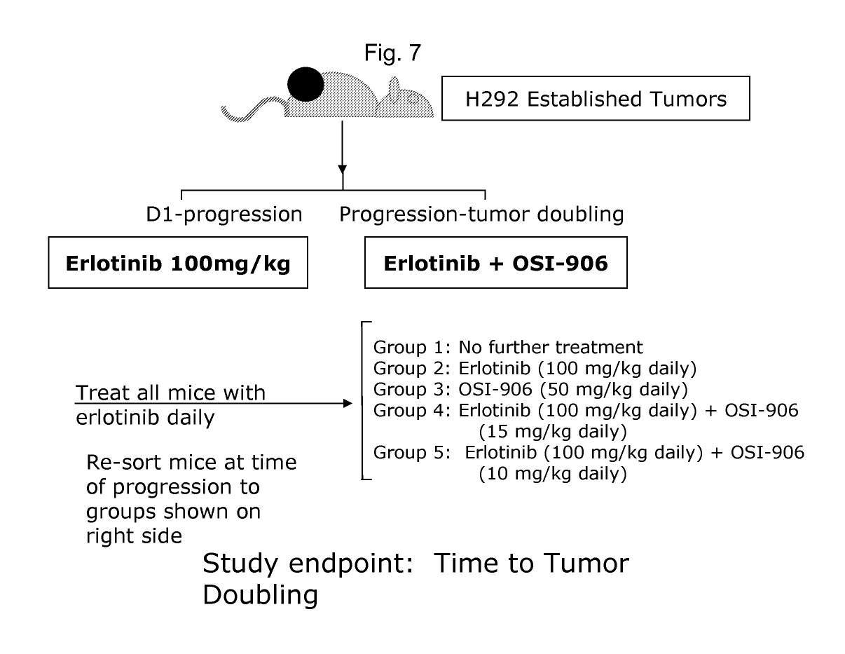

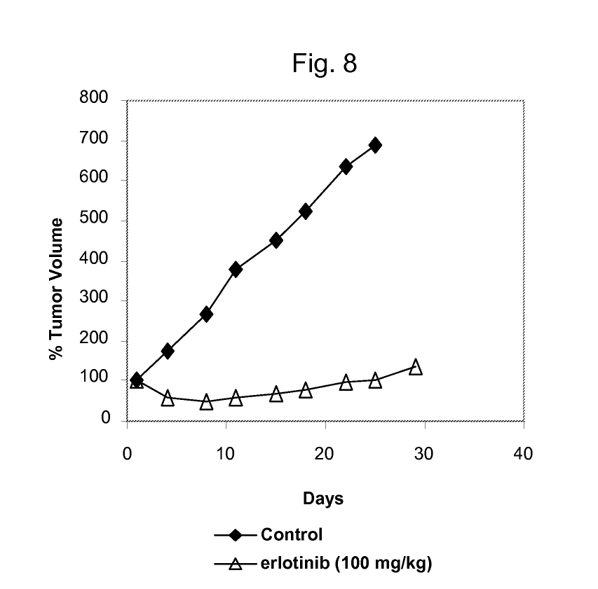

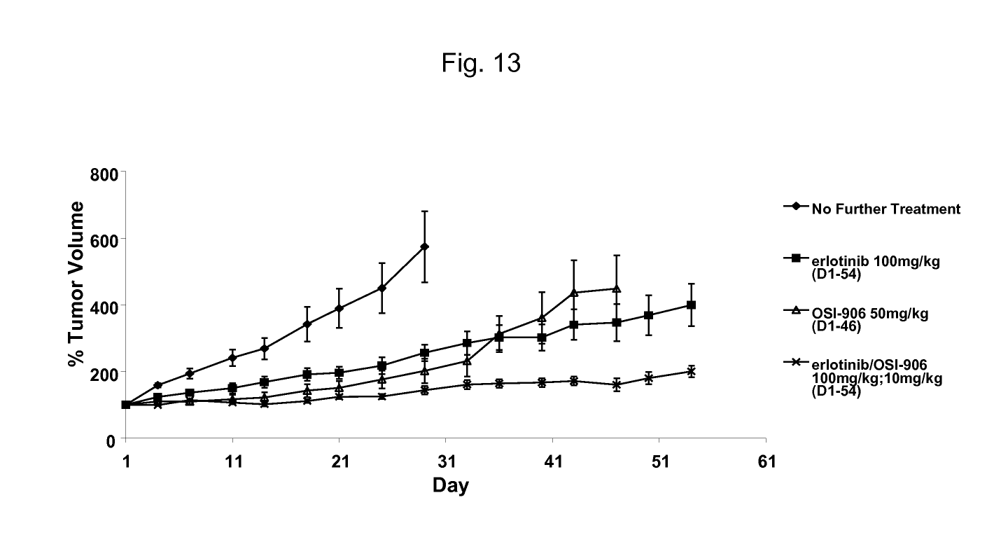

The schematic illustrating the TBP study in the H292 model is shown in FIG. 7. Following 28 days of oral dosing with erlotinib (100 mg/kg per day, see FIG. 8), mice that initially responded to erlotinib (demonstrated tumor growth inhibition as described above) and then started to have tumor regrowth (progression) while still on erlotinib treatment were randomly re-sorted into one of the following groups, n=8 per group: 1) no further treatment, 2) maintenance of erlotinib treatment, 3) taken off erlotinib and put on OSI-906 treatment (50 mg/kg per day), 4) maintained on erlotinib treatment and had OSI-906 (15 mg/kg per day) added to the treatment regimen, and 5) maintained on erlotinib treatment and had OSI-906 (10 mg/kg) added to the treatment regimen. See FIGS. 9 and 13.

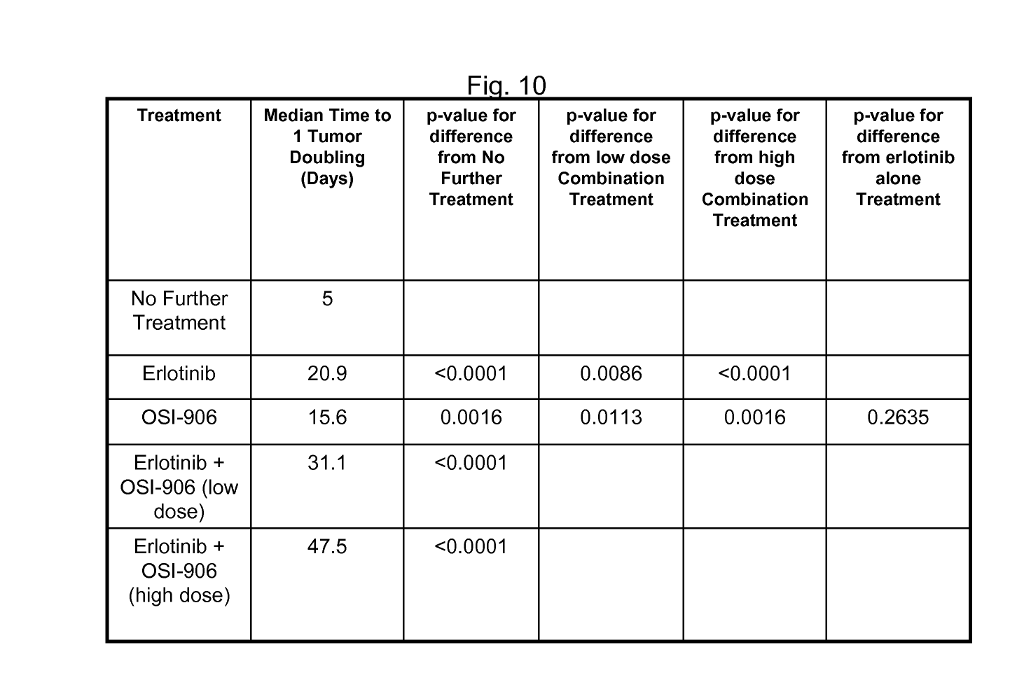

FIGS. 10 (and 14) and 11 (and 15) show statistical analysis of the data for the time to one and two tumor size doublings, respectively.

For example, in FIG. 10, it is shown, among other things, that the median time to one doubling was extended to a statistically significant degree by Kaplan-Meier Analysis from 20.9 days (erlotinib) to 31.1 or 47.5 days (combination TBP regimens).

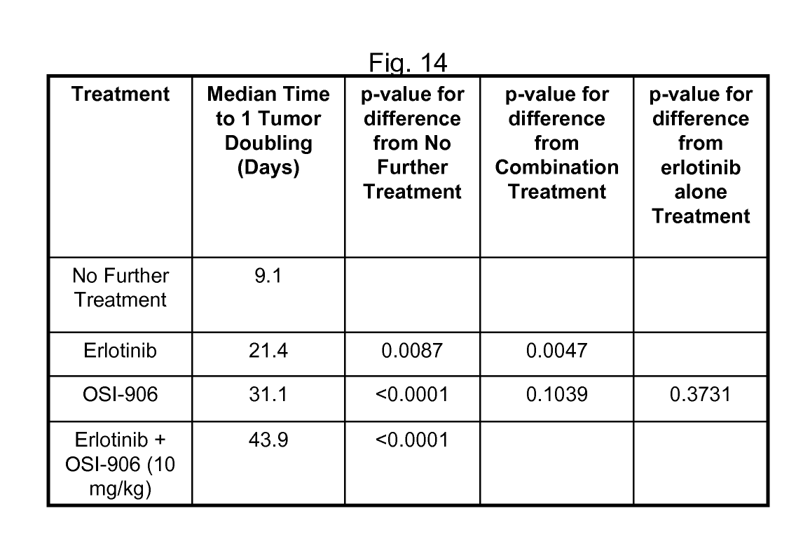

In FIG. 14, it is shown, among other things, that the median time to one doubling was extended to a statistically significant degree by Kaplan-Meier Analysis from 20.9 days (erlotinib) to 43.9 days (combination TBP regimens).

The addition of OSI-906 to erlotinib treatment at the time of progression on erlotinib results in statistically significant prolonged time to tumor doubling. There was no statistical difference between maintaining erlotinib single agent treatment and switching to OSI-906 single agent treatment.

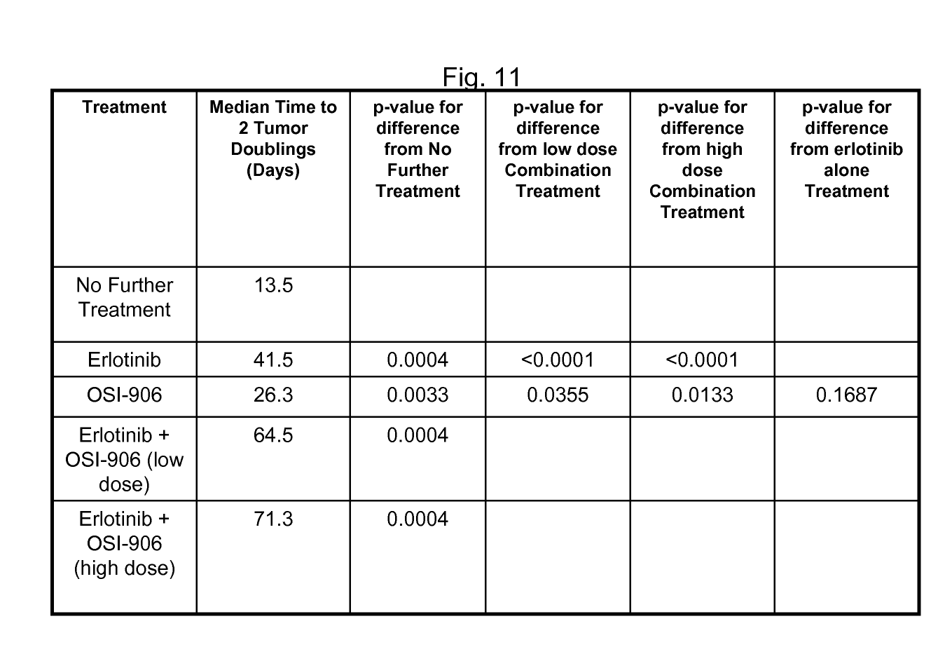

In FIG. 11, it is shown, among other things, that the median time to two doublings was also extended to a statistically significant degree by Kaplan-Meier Analysis from 41.5 days (erlotinib) to 64.5 or 71.3 days (combination TBP regimens).

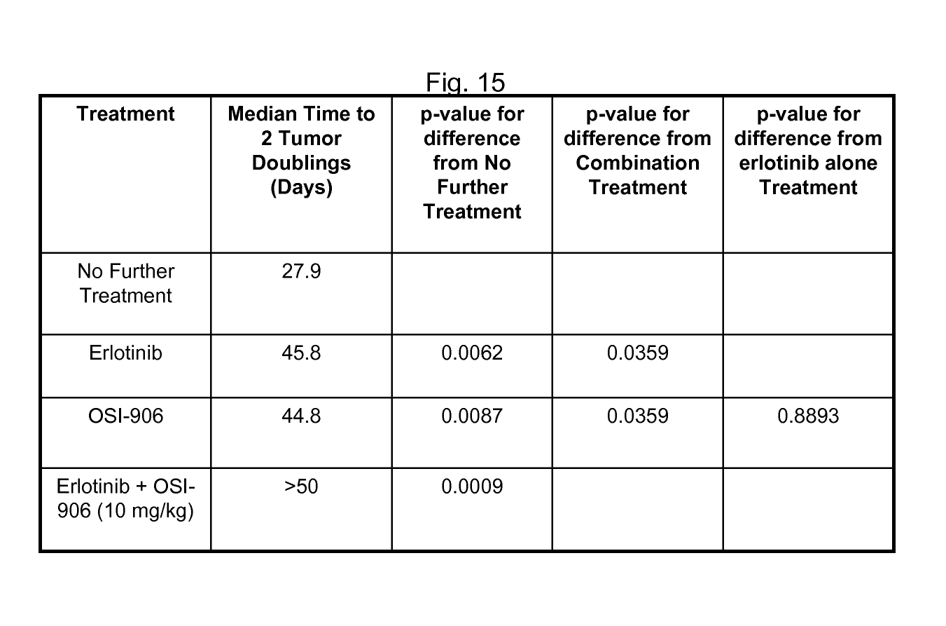

In FIG. 15, it is shown, among other things, that the median time to two doublings was also extended to a statistically significant degree by Kaplan-Meier Analysis from 45.8 days (erlotinib) to >50 days (combination TBP regimens).

The addition of OSI-906 to erlotinib treatment at the time of progression on erlotinib results in statistically significant prolonged time to two tumor doublings. There was no statistical difference between maintaining erlotinib single agent treatment and switching to OSI-906 single agent treatment.

### Frontline Combination Treatment—H292 Model

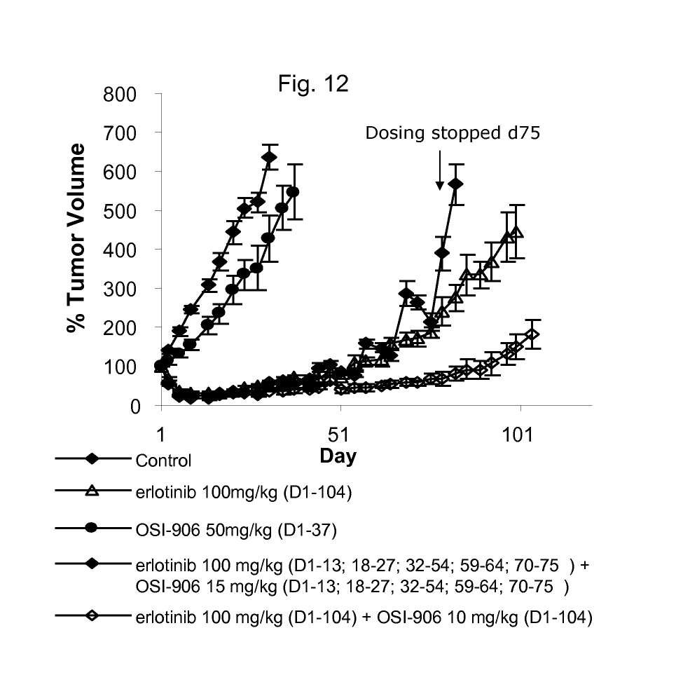

FIG. 12 shows the dosing and results of a study of OSI-906 and erlotinib frontline combination regimen in the H292 model described above. By about 51 days, tumor volume separation between combination and erlotinib-only arms is observed. The dosing was cycled in the erlotinib+15 mg/kg OSI-906 arm due to observed body weight loss and then was discontinued after 75 days. By 100 days, 7 of 8 erlotinib only mice doubled their tumor volumes twice and only 4 of 8 mice on the erlotinib+10 mg/kg OSI-906 dosing regimen doubled their tumor volume once.

### DEFINITIONS

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

The term “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

“Tumor growth” or “tumor metastases growth”, as used herein, unless otherwise indicated, is used as commonly used in oncology, where the term is principally associated with an increased mass or volume of the tumor or tumor metastases, primarily as a result of tumor cell growth.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient. The term “treatment” as used herein, unless otherwise indicated, refers to the act of treating.

The phrase “a method of treating” or its equivalent, when applied to, for example, cancer refers to a procedure or course of action that is designed to reduce or eliminate the number of cancer cells in an animal, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an animal, is nevertheless deemed an overall beneficial course of action.

The term “therapeutically effective agent” means a composition that will elicit the biological or medical response of a tissue, system, animal or human that is being sought by the researcher, veterinarian, medical doctor or other clinician.

The term “therapeutically effective amount” or “effective amount” means the amount of the subject compound or combination that will elicit the biological or medical response of a tissue, system, animal or human that is being sought by the researcher, veterinarian, medical doctor or other clinician.

As used herein, the term “patient” preferably refers to a human in need of treatment with an IRS1 agent such as an EGFR kinase inhibitor for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an IRS1 agent.

For purposes of the present invention, “co-administration of” and “co-administering” of an IGF1R protein kinase inhibitor compound of Formula I with an IRS1 agent such as an EGFR kinase inhibitor (both components referred to hereinafter as the “two active agents”) refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. An IGF1R protein kinase inhibitor compound of Formula I can be administered prior to, at the same time as, or subsequent to administration of the IRS1 agent, or in some combination thereof. Where the IRS1 agent is administered to the patient at repeated intervals, e.g., during a standard course of treatment, an IGF1R protein kinase inhibitor compound of Formula I can be administered prior to, at the same time as, or subsequent to, each administration of the IRS1 agent, or some combination thereof, or at different intervals in relation to the IRS1 agent treatment, or in a single dose prior to, at any time during, or subsequent to the course of treatment with the IRS1 agent.

The term “refractory” as used herein is used to define a cancer for which treatment (e.g. chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease).

Abbreviations: EGF, epidermal growth factor; EGFR, epidermal growth factor receptor; EMT, epithelial-to-mesenchymal transition; MET, mesenchymal-to-epithelial transition; NSCL, non-small cell lung; NSCLC, non-small cell lung cancer; HNSCC, head and neck squamous cell carcinoma; CRC, colorectal cancer; MBC, metastatic breast cancer; Brk, Breast tumor kinase (also known as protein tyrosine kinase 6 (PTK6)); LC, liquid chromatography; IGF-1, insulin-like growth factor-1; TGFα, transforming growth factor alpha; IC₅₀, half maximal inhibitory concentration; pY, phosphotyrosine; wt, wild-type; PI3K, phosphatidyl inositol-3 kinase; GAPDH, glyceraldehyde 3-phosphate dehydrogenase; MAPK, mitogen-activated protein kinase; PDK-1,3-Phosphoinositide-Dependent Protein Kinase 1; Akt, also known as protein kinase B, is the cellular homologue of the viral oncogene v-Akt; mTOR, mammalian target of rapamycin; 4EBP1, eukaryotic translation initiation factor-4E (mRNA cap-binding protein) Binding Protein-1, also known as PHAS-I; p70S6K, 70 kDa ribosomal protein-S6 kinase; eIF4E, eukaryotic translation initiation factor-4E (mRNA cap-binding protein); Raf, protein kinase product of Raf oncogene; MEK, ERK kinase, also known as mitogen-activated protein kinase; ERK, Extracellular signal-regulated protein kinase, also known as mitogen-activated protein kinase; PTEN, “Phosphatase and Tensin homologue deleted on chromosome 10”, a phosphatidylinositol phosphate phosphatase; pPROTEIN, phospho-PROTEIN, “PROTEIN” can be any protein that can be phosphorylated, e.g. EGFR, ERK, S6 etc; PBS, Phosphate-buffered saline; TGI, tumor growth inhibition; WFI, Water for Injection; SDS, sodium dodecyl sulfate; ErbB2, “v-erb-b2 erythroblastic leukemia viral oncogene homolog 2”, also known as HER-2; ErbB3, “v-erb-b2 erythroblastic leukemia viral oncogene homolog 3”, also known as HER-3; ErbB4, “v-erb-b2 erythroblastic leukemia viral oncogene homolog 4”, also known as HER-4; FGFR, Fibroblast Growth Factor Receptor; DMSO, dimethyl sulfoxide.

All patents, published patent applications and other references disclosed herein are hereby expressly incorporated herein in their entireties by reference.

1. A method of treating cancer comprising:
(a) selecting a patient having cancer that responded to a first EGFR inhibitor therapy and that has resumed progression;
(b) administering to the patient an effective regimen comprising (i) at least one second EGFR inhibitor and (ii) at least one IGF-1R inhibitor;
wherein the at least one second EGFR inhibitor is the same or different as the agent(s) used in the first EGFR inhibitor therapy, and;
wherein (i) and (ii) are administered together or sequentially.

2. The method of claim 1, wherein the IGF-1R inhibitor comprises OSI-906 or a pharmaceutically acceptable salt thereof.

3. The method of claim 2, wherein the OSI-906 is administered in an amount of about 0.1 to about 0.7 mg/kg·day.

4. The method of claim 2, wherein the OSI-906 is administered in an amount of about 0.7 to about 5 mg/kg·day.

5. The method of claim 2, wherein the OSI-906 is administered in an amount of about 5 to about 15 mg/kg·day.

6. The method of claim 2, wherein the second EGFR inhibitor comprises a small-molecule non-biologic agent.

7. The method of claim 2, wherein the second EGFR inhibitor comprises erlotinib or a pharmaceutically acceptable salt thereof.

8. The method of claim 2, wherein the second EGFR inhibitor comprises a monoclonal antibody.

9. The method of claim 2, wherein the second EGFR inhibitor comprises gefitinib, CI-1033, cetuximab, panitumumab, lapatinib, lapatinib ditosylate, ZACTIMA™, BMS-599626, ARRY-334543, or AG490.

10. The method of claim 7, wherein the second EGFR inhibitor comprises same agent(s) used in the first EGFR inhibitor therapy.

11. The method of claim 7, further comprising administering at least one additional active agent.

12. The method of claim 7, wherein the cancer comprises NSCLC.

13. The method of claim 7, wherein the cancer comprises pancreatic cancer, head and neck cancer, breast cancer, ACC, or neuroblastoma.

14. The method of claim 7, wherein the IGF-1R and second EGFR inhibitors are administered together.

15. The method of claim 7, wherein the IGF-1R and second EGFR inhibitors behave synergistically.

16. The method of claim 7, wherein the IGF-1R and second EGFR inhibitors behave additively.

17. The method of claim 7, wherein the patient exhibits progression-free survival for at least about eight weeks from the administration of the first dose of the IGF-1R inhibitor.

18. The method of claim 7, wherein the patient exhibits progression-free survival for at least about sixteen weeks from the administration of the first dose of the IGF-1R inhibitor.

19. An oral pharmaceutical composition comprising OSI-906 and erlotinib or pharmaceutically acceptable salts thereof, formulated with or without at least one pharmaceutically acceptable carrier.
